(19) United States
(12) Patent Application Publication
(10) Pub. No.: US 2026/0048226 A1
(43) Pub. Date: Feb. 19, 2026

(54) DIGITALLY ENABLED, PRESSURE SWING ADSORPTION-BASED INTERMITTENT HYPOXIA-HYPEROXIA TRAINING SYSTEMS

(71) Applicant: L-Newco, Inc. (DBA Vitaliti), Hillsborough, CA (US)

(72) Inventors: Denis Varvanets, Los Gatos, CA (US); Stephen Price, Spring, TX (US); Mahmood Panjwani, Hillsborough, CA (US); Ganga Nadella, Menlo Park, CA (US)

(21) Appl. No.: 19/304,356

(22) Filed: Aug. 19, 2025

Related U.S. Application Data
(60) Provisional application No. 63/684,748, filed on Aug. 19, 2024.

(51) Int. Cl. A61M 16/10; A61M 16/20

(52) U.S. Cl. CPC A61M 16/101; A61M 16/105; A61M 16/202; A61M 16/209; A61M 2205/3303; A61M 2205/3334; A61M 2205/3584; A61M 2205/50

(57) ABSTRACT

A device for intermittent hypoxia-hyperoxia training includes a main chassis; a pressure swing adsorption (PSA) system housed within the chassis, the PSA system configured to generate a hyperoxic gas output and a hypoxic gas output; a buffering reservoir housed within the main chassis, configured to receive the hypoxic gas output from the PSA system; a single user output port; a valve mechanism having a first inlet connected to an output of the buffering reservoir, a second inlet connected to the hyperoxic gas output of the PSA system, and an outlet connected to the single user output port; and a computation unit configured to control the valve mechanism, thereby selecting whether hypoxic gas from the buffering reservoir or hyperoxic gas from the PSA system is delivered to the single user output port.

### CROSS-REFERENCE TO RELATED APPLICATIONS

This application claims priority to U.S. Provisional Patent Application No. 63/684,748, filed Aug. 19, 2024, entitled “AI-Powered Intermittent Hypoxia-Hyperoxia Training Device,” the entire content of which is incorporated by reference herein.

### TECHNICAL FIELD

The present disclosure generally relates to intermittent hypoxia-hyperoxia training (IHHT) systems, and more specifically, to neurovascularly guided control of IHHT training using multi-modal physiological sensing, coupled to a zeolite-based digitally controlled pressure swing adsorption system with a single-mask output and an integrated, internal gas buffer.

### BACKGROUND OF THE DISCLOSURE

Intermittent hypoxia-hyperoxia training alternates reduced-oxygen (hypoxia) and elevated-oxygen (hyperoxia) breathing periods to elicit adaptations in mitochondrial efficiency, autonomic balance, and cardiorespiratory performance. Conventional IHHT implementations typically follow preprogrammed schedules (e.g., fixed-length hypoxic bouts followed by fixed recovery periods) that are not personalized to a user's moment-to-moment physiological state. In such systems, the delivered dose is primarily defined by time at a target gas composition rather than by the user's actual physiological response.

Existing control strategies often rely on a single parameter, most commonly peripheral blood oxygen saturation (SpO₂), to gate or terminate hypoxic intervals. While SpO₂ is useful, it can exhibit lag relative to arterial oxygen dynamics and may not reflect neuro-autonomic load or metabolic stress during or after a hypoxic exposure. As a result, two individuals with identical SpO₂ traces can experience markedly different autonomic activation and glycemic excursions, leading to inconsistent outcomes and potential safety concerns when using uniform protocols.

From a hardware perspective, many IHHT devices generate one conditioned gas stream and blend with ambient air to approximate hypoxic or hyperoxic conditions, or they rely on membrane-based separation that limits achievable hyperoxic purity. Transitions between gas states can be slow, and some systems require users to swap masks or external hoses when switching phases. To smooth flow, a number of devices employ external inflatable buffer bags that increase footprint, introduce potential leak/failure points, complicate sanitation, and add compliance that can degrade the responsiveness of closed-loop control.

These architectural choices create specific technical constraints: (i) limited peak fraction of inspiration O₂ (FiO₂) for short, protective hyperoxic bursts; (ii) sluggish phase switching that reduces the precision of dose delivery; and (iii) transport and compliance delays introduced by long external circuits and bags, which blur the temporal alignment between sensor-detected events and controller actions. Due to the sluggish phase switching, transport and compliance delays, and other technical constraints, dynamic control of the IHHT devices during treatments becomes infeasible or extremely challenging.

Further, personalization remains underdeveloped in the existing IHHT devices. Real-time autonomic markers such as heart-rate variability (HRV) provide insight into sympathetic/parasympathetic balance, blood pressure (BP) is the hemodynamic guardrail, perfusion index (PI) is both a signal-quality sentinel and a physiologic window into peripheral vasomotor tone, while continuous glucose monitoring (CGM) reflects metabolic stress and recovery kinetics. Conventional systems generally do not fuse these signals, and therefore cannot obtain a live gauge of how hard the nervous system is working to cope with the stimulus, and cannot enforce individualized “metabolic guardrails,” such as limiting hypoxic dose when glucose excursion or recovery slope exceeds user-specific thresholds. Data fusion for IHHT presents its own challenges: signals arrive at different cadences and latencies (e.g., interstitial glucose lag relative to blood, HRV requiring windowed estimation, motion artifacts affecting photoplethysmography).

The foregoing examples of the related art and limitations therewith are intended to be illustrative and not exclusive, and are not admitted to be “prior art.” Other limitations of the related art will become apparent to those of skill in the art upon a reading of the specification and a study of the drawings.

### SUMMARY OF THE DISCLOSURE

The present disclosure addresses the above-mentioned problems and other problems in the existing IHHT devices by providing artificial intelligence (AI)-powered IHHT devices with protocols that can be personalized in advance and adapted in actual training processes.

In one aspect, an apparatus for intermittent hypoxia-hyperoxia training includes: a main chassis; a pressure swing adsorption (PSA) system housed within the chassis, the PSA system configured to generate a hyperoxic gas output and a hypoxic gas output; a buffering reservoir housed within the main chassis, configured to receive the hypoxic gas output from the PSA system; a single user output port; a valve mechanism having a first inlet connected to an output of the buffering reservoir, a second inlet connected to the hyperoxic gas output of the PSA system, and an outlet connected to the single user output port; and a computation unit configured to control the valve mechanism, thereby selecting whether hypoxic gas from the buffering reservoir or hyperoxic gas from the PSA system is delivered to the single user output port.

In another aspect, a method, for providing adaptive IHHT training using the apparatus described elsewhere, includes: receiving, at the external user device, a plurality of real-time physiological data streams from one or more wearable sensors monitoring a user; processing, by the external user device, said physiological data streams to determine a need for a protocol adjustment; transmitting, from the external user device to the computation unit of the apparatus via the wireless transceiver, a command to implement the protocol adjustment; and executing, by the computation unit, the command to adjust the training being administered to the user.

In another aspect, a system for IHHT training includes: one or more sensors configured to obtain physiological data of a user under the IHHT training; a computation unit configured to compute a score based on the physiological data of the user; a gas generator produce a nitrogen-enriched, oxygen-reduced hypoxic gas stream and an oxygen-enriched hyperoxic gas stream, where the gas generator is further fluidly connected to a first buffering reservoir for buffering the hyperoxic gas stream and a second buffering reservoir for buffering the hypoxic gas stream; a computer-controlled switching valve fluidically coupled to the first buffering reservoir and the second reservoir; and a single breathing interface coupled to the switching valve, where the computation unit further includes a controller operatively coupled to the switching valve to adjust a flow of at least one of the hyperoxic gas stream or the hypoxic gas stream during a training session responsive to the generated score.

The foregoing is a summary and thus contains, by necessity, simplifications, generalizations, and omissions of detail; consequently, the summary is illustrative only and is not limiting in any way. Other aspects, inventive features, and advantages of the systems and/or processes described herein may become apparent in the non-limiting detailed description set forth herein.

### BRIEF DESCRIPTION OF THE DRAWINGS

These and other features, aspects, and advantages of the present disclosure may become better understood with regard to the following description, and accompanying drawings, where:

Figure (FIG.) 1 is a schematic diagram of an AI-powered intermittent hypoxia-hyperoxia training device, in accordance with some embodiments.

FIG. 2 shows example system components for an AI-powered intermittent hypoxia-hyperoxia training device, in accordance with some embodiments.

FIG. 3 shows an example digitally controlled pressure swing adsorption system inside an AI-powered intermittent hypoxia-hyperoxia training device, in accordance with some embodiments.

FIG. 4 illustrates an example control of an AI-powered intermittent hypoxia-hyperoxia training device through a mobile device, in accordance with some embodiments.

FIG. 5 shows an example accordion-style reservoir for an internal buffering reservoir of an AI-powered intermittent hypoxia-hyperoxia training device, in accordance with some embodiments.

FIG. 6 shows an example process flow for an AI-powered intermittent hypoxia-hyperoxia training session, in accordance with some embodiments.

FIG. 7 shows an example computing device for implementing systems and methods described in reference to FIGS. 1-6.

### DETAILED DESCRIPTION

To make the aforementioned objects, features, and advantages of the present disclosure more obvious and understandable, the present disclosure may be further described below with reference to the accompanying drawings and embodiments.

It should be noted that specific details are set forth in the following description to fully understand the present disclosure. However, the present disclosure may be implemented in many other ways different from those described herein, and those skilled in the art may make similar generalizations without violating the connotation of the present disclosure. Therefore, the present disclosure is not limited by the specific embodiments disclosed below.

The terms used in the embodiments of the present disclosure are only for the purpose of describing specific embodiments and are not intended to limit the present disclosure. The singular forms of “a”, “said”, and “the” used in the embodiments of the present disclosure and the appended claims are also intended to include plural forms unless the context clearly indicates other meanings.

It should be noted that the example embodiments may be implemented in various forms, and should not be construed as being limited to the embodiments set forth herein. On the contrary, the provision of these embodiments makes the present disclosure more comprehensive and complete, and fully conveys the concept of the example embodiments to those skilled in the art. The same reference numerals in the figures indicate the same or similar structures, and thus their repeated description may be omitted. In addition, the similarities between the embodiments may not be repeated.

### Motivation and Benefits

The disclosure is motivated by the need to deliver IHHT in a way that is (i) mechanically capable of rapid, precise transitions without bulky external components, and (ii) physiologically individualized in real time. Legacy hardware (single stream with blending, external buffer bags) slows transitions, increases leakage/failure points, and complicates sanitation and transport, which makes the conventional time-based protocols and single-parameter control yield variable outcomes and safety tradeoffs.

The benefits of the disclosed system follow from a hardware update that enables responsiveness that matches the temporal resolution of the control logic. A digitally controlled pressure swing adsorption (PSA) system and a fast switching valve produce both hypoxic and high-purity hyperoxic streams and transition between them with minimal delay. An internal buffer reduces transport and compliance lag relative to external bags and long circuits, improving the alignment between sensed physiological events and delivered gas composition and thereby sharpening the effective dose profile.

By computing a unified neurovascular and/or stress score from SpO₂, HRV, blood pressure, perfusion index, and CGM, the controller also titrates hypoxic and hyperoxic intervals to the individual in real time due to the updated hardware, reducing both under- and over-dosing and stabilizing outcomes across sessions. Personalized “metabolic guardrails”, such as limiting hypoxic exposure when glucose excursions or recovery slope exceed user-specific thresholds, further improve safety while preserving therapeutic effect, and automatic hyperoxic rescue bursts provide additional protection when the model detects excessive load.

Usability and reliability are enhanced by routing both phases through a single patient interface and by integrating the buffer within the chassis. Eliminating external inflatable bags reduces footprint and setup steps, removes common leak and failure points, and simplifies cleaning and sanitation, all of which support consistent field operation in clinical and wellness settings.

The system also produces measurable, longitudinal biomarkers to guide programming. A standardized hypoxic challenge paired with CGM excursion and recovery kinetics yields a biomarker of metabolic flexibility that can inform clinician oversight and program tuning. Wireless sensor ingress and cloud reporting support remote monitoring and software updates, while faster transitions reduce session length and staff interventions. The zeolite-based architecture avoids membrane supply constraints and lowers consumables, improving deployment economics.

It is to be noted that the benefits and advantages described herein are not all-inclusive, and many additional features and advantages will be further described under the context of specific embodiments. In addition, some additional features and advantages will become apparent to one of ordinary skill in the art in view of the figures and the following descriptions.

### Overall System

In the following, an AI-powered IHHT device 100 is described with reference to specific hardware components and/or software components. As illustrated in FIG. 1, exemplary hardware components may include: (a) a sensor suite 110 including one or more of SpO₂ sensor, an HRV sensor (e.g., electrocardiogram (ECG) or photoplethysmography (PPG)-derived), a blood pressor sensor, a CGM sensor, or other different physiological sensors; (b) a computation unit 120 with processors, memory, and storage executing a trained model included therein; (c) a digitally controlled PSA system 130; (d) a buffering reservoir 140 comprising an internal, chassis-integrated reservoir (which itself may be a part of the digitally controlled PSA system); (e) a controller 150 driving valves, flow, and FiO₂; and wireless interfaces (e.g., Bluetooth, ANT+) (not shown) for sensor ingress and/so for possible other wireless connections, such as connecting to a user's mobile phone or a remote monitoring device. All of these components are internal components inside a chassis 105, where a single breathing interface (mask or nasal interface) 160 is connected. In some embodiments, the IHHT device 100 may further include or be coupled to a mobile and/or local UI, secure storage, and cloud services for remote monitoring, clinician dashboards, and model updates. In some embodiments, the IHHT device 100 is packaged in a carry-on-sized, wheeled chassis 105 with a retractable handle and a total mass under a certain value (e.g., under 10 kg, 15 kg, 20 kg, 30 kg, 40 kg, 50 kg, 60 kg, etc.), enabling one-handed transport like a suitcase between rooms or sites.

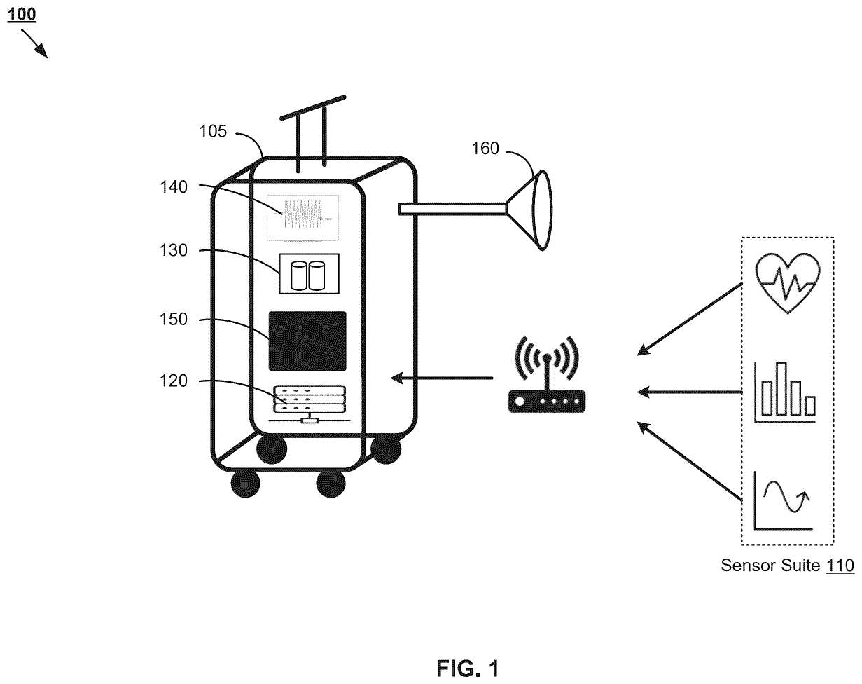

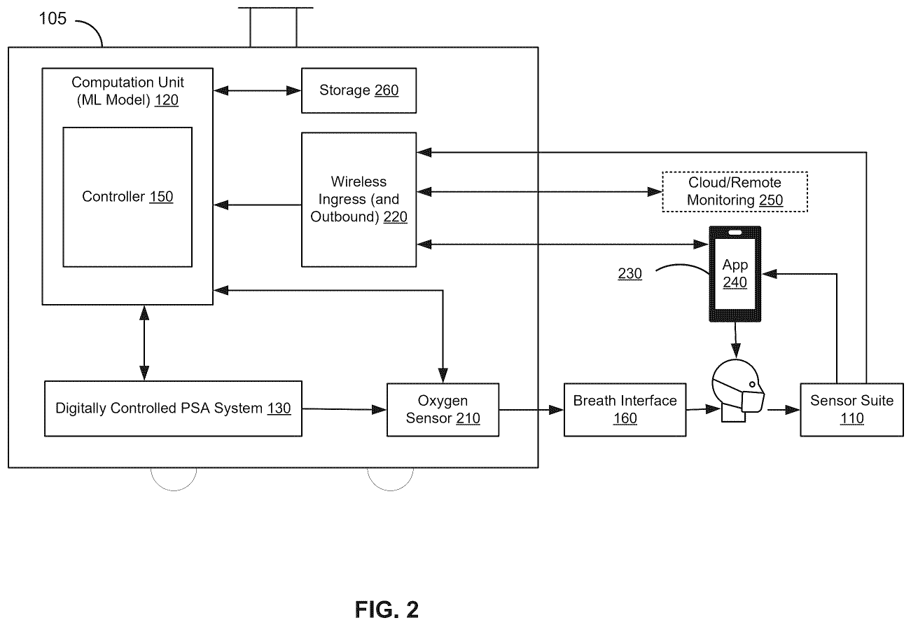

In operation, briefly, the digitally controlled PSA system 130 may provide necessary oxygen levels, which are provided to the breathing interface 160 through the buffering reservoir 140 that smooths flow to the breathing interface, while safety monitors enforce guardrails. At the same time, sensor data from the sensor suite 110 is collected, which then arrives at the computation unit 120/controller 150 via wireless ingress, which drives the controller 150 to dynamically adjust FiO₂ targets and interval timing. For example, the controller 150 may actuate the switching valve to select between the hypoxic gas stream and the hyperoxic gas stream provided by the PSA system 130. The operation of the disclosed IHHT device 100 is further described in detail with reference to specific components in FIG. 2.

### Specific Components in the IHHT Device

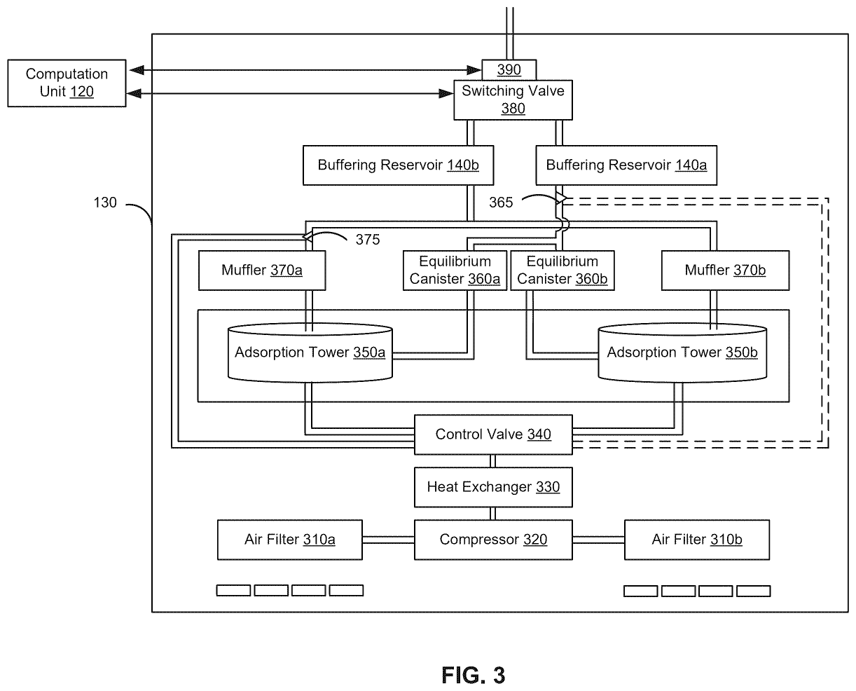

The digitally controlled PSA system 130 may condition ambient air (filtration and compression) and perform gas separation (e.g., via pressure-swing adsorption and/or controlled dilution) to produce a nitrogen-enriched, oxygen-reduced stream at a commanded FiO₂ for hypoxic phases (also referred to as hypoxic gas stream), as well as an oxygen-enriched stream at a commanded purity (e.g., FiO₂ at 80% or higher) (also referred to as hyperoxic gas stream). It may regulate gas delivery using a product volume to stabilize flow and pressure, verify composition with an oxygen (or proxy) sensor 210, and isolate its path with one-way valving to prevent cross-flow. In some embodiments, certain control interfaces may be further included to allow the controller 150 to set and/or further adjust targets, ramps, and limits (e.g., FiO₂, cycle timing, purge ratio). In some embodiments, safety logic may be further included to enforce guardrails (e.g., minimum breathable state on fault, leak/occlusion detection) and bias the circuit to a safe condition upon error or power loss, before handing the hypoxic output to the blender/switching stage. The specific details for the digitally controlled PSA system are further described in FIG. 3.

The oxygen sensor 210 is disposed between the buffering reservoir and the user's breathing mask. The buffering reservoir in the digitally controlled PSA system 130 stabilizes the airflow, but the actual gas stream the user inhales can vary depending on many factors, e.g., generator efficiency, switching valve timing, leaks, or even backflow from the user's exhalation. By positioning the oxygen sensor at this final stage, just before the mask, the device measures the true FiO₂ that reaches the user. Accordingly, the oxygen sensor 210 ensures that the oxygen concentration delivered is exactly what the AI-controlled protocol intends, rather than relying on theoretical generator output. If the oxygen level at the mask falls below or rises above set safety thresholds (e.g., below a hypoxia floor or above a hyperoxia ceiling), the system may also trigger emergency hyperoxic bursts or even shut down delivery. In addition, because the sensor measures actual flow dynamics at the end of the circuit, discrepancies between commanded and measured values reveal leaks, blockages, or disconnections. Further, as a critical part of the closed-loop control, data from the oxygen sensor 210 feeds directly into the computation unit, which uses it along with physiological sensors (SpO₂, HRV, CGM, blood pressure, perfusion index) to continuously adapt protocol intensity in real time. It should be noted that, under certain circumstances, an oxygen sensor might not be required in actual applications. Instead, FiO₂ levels may be determined through a variety of pressure sensors installed along the pathway without using an oxygen sensor.

The breathing interface 160 may provide a single patient interface (e.g., full-face mask, nasal mask, or nasal cannula) for both hypoxic and hyperoxic phases without component swaps. It couples to the outlet of the digitally controlled PSA system 130 and maintains a comfortable, low-leak seal while minimizing dead space and inspiratory resistance. An exhalation pathway (e.g., integrated vent or valve) may be further included to manage CO₂ washout, and an optional sampling port may be included to support end-tidal or line pressure sensing. In some embodiments, the breathing interface 160 may include quick-release fittings, multiple sizes and seal geometries for fit, and biocompatible/cleanable materials suitable for repeated use.

The sensor suite 110 may provide multi-modal physiological inputs for closed-loop IHHT. In some embodiments, the suite includes at least one of: (i) a blood oxygen saturation (SpO₂) sensor; (ii) an HRV source obtained from ECG or PPG; and (iii) a continuous glucose monitor. Additional optional channels may include, but are not limited to, respiration (e.g., flow/pressure or impedance), end-tidal CO₂, perfusion index, skin or gas temperature, ambient pressure and humidity, and user-context sensors such as a 3-axis accelerometer/gyroscope. Sensors may be integrated on-device, connected via wired ports, or paired wirelessly (e.g., through wireless ingress 104), which are not limited in the present disclosure.

A blood oxygen saturation sensor measures the percentage of hemoglobin in the blood that is saturated with oxygen, typically expressed as SpO₂. Implemented as a pulse oximeter, a SpO₂ ring-style oximeter, or other proper formats, the blood oxygen saturation sensor uses light-based technology, emitting red and infrared wavelengths through body tissue (e.g., a fingertip or earlobe) and detecting how much light is absorbed. Since oxygenated and deoxygenated hemoglobin absorb light differently, the sensor may calculate oxygen saturation by analyzing the absorption ratio. In the IHHT training, SpO₂ readings are essential for tracking the hypoxic and hyperoxic phases, ensuring safety, and guiding the adaptive control algorithms.

Heart-rate variability refers to the variation in the time interval between successive heartbeats, a key indicator of autonomic nervous system balance and stress resilience. HRV may be derived from two main sensor modalities. The first one is ECG, which measures the electrical activity of the heart via skin electrodes, providing precise detection of the R-R intervals (the time between successive R-waves in the cardiac cycle). ECG-derived HRV is considered the gold standard for accuracy. The second one is PPG, which uses optical sensors (which may be in wearables like smartwatches) to detect blood volume changes in the microvascular bed of tissue. While less precise than ECG, PPG-based HRV is more convenient and may be collected from devices like finger clips or wristbands. In the IHT training, HRV may serve as a measure of the body's autonomic response to hypoxic stress, complementing SpO₂ data in the AI's feedback loop.

Blood pressure is the hemodynamic guardrail. Hypoxia can raise blood pressure via sympathetic activation and peripheral vasoconstriction, but the magnitude and tolerance vary widely by individual and comorbidities. Using absolute ceilings (e.g., clinician-defined stop criteria) and relative changes from a personal baseline lets a user prevent unsafe cardiovascular strain while still delivering an effective stimulus. If a user has continuous beat-to-beat BP, slow-trend monitoring can catch rising afterload or exaggerated pressor responses; with intermittent cuff readings, pre-/mid-/post-session checks still reveal problematic deltas. BP also helps discriminate benign tachycardia from risky responses: a modest heart-rate rise with stable pressures is very different from a large systolic blood pressure (SBP)/diastolic blood pressure (DBP) jump or a sudden drop, suggesting vasovagal features. When BP breaches limits, the controller should extend recovery, reduce the next hypoxic window, or end the session.

Perfusion index is both a signal-quality sentinel and a physiologic window into peripheral vasomotor tone. Computed from the photoplethysmogram as the pulsatile-to-nonpulsatile ratio, PI plummets with cold extremities, sensor misplacement, or motion, exactly the conditions that make SpO₂ numbers untrustworthy. Using PI to gate decisions prevents the controller from chasing artifacts: if PI is below a quality threshold or drops abruptly, a user can pause closed-loop adjustments, prompt a sensor check, or switch sites. When PI is high-quality and stable, its physiologic changes add context: a sustained PI drop during hypoxia (with good signal) indicates sympathetic-driven vasoconstriction and rising autonomic load, supporting HRV-based down-titration even if SpO₂ looks acceptable. In short, PI ensures a user acts on the clean data first, and then doubles as a sensitive indicator of peripheral stress once the data are clean.

Furthermore, a continuous glucose monitor tracks glucose levels in interstitial fluid in near real-time, e.g., using a minimally invasive sensor inserted just under the skin. The sensor may measure glucose concentrations via enzymatic electrochemical reactions, and a transmitter sends this data wirelessly to a receiver or mobile device. CGMs provide glucose readings every few minutes, revealing trends and fluctuations throughout the day. In an IHHT setting, CGM data may add a metabolic dimension to protocol control, showing how the body responds to hypoxic stress in terms of glucose mobilization and clearance, and enabling a “neuro-metabolic guided” training approach, which is a control methodology that adapts therapy parameters in real time based on both autonomic nervous system signals (neuro) and metabolic state indicators (metabolic).

Wireless ingress 220 in the IHHT device refers to its ability to receive physiological data from external sensors 110 via wireless communication protocols such as Bluetooth or ANT+. This feature allows wearable sensors, like SpO₂ monitors, HRV trackers, and continuous glucose and blood pressure monitors, to transmit their readings directly to the computation unit 120 (which may be a part of the controller or coupled to the controller 150) without requiring physical cables. By eliminating wired connections, wireless ingress 220 enhances user comfort, supports unrestricted movement during training, and simplifies setup, making the device more suitable for both home and clinical environments. In some embodiments, the wireless ingress 220 receives the physiological data directly from the external sensors. In other embodiments, the wireless ingress 220 may receive the physiological data from a mobile device 230 associated with the user, where the mobile device 230 may include a mobile application 240 configured for monitoring signal collection and wireless transmission to the IHHT device. In some embodiments, the same or different application 240 on the mobile phone may further enable the user to select a training protocol, view and monitor the training session, modify or adjust protocol settings, view real-time data during the training session, review reports and/or analysis after the training session, etc.

Beyond convenience, wireless ingress 220 is critical for the system's real-time adaptive capabilities. In a neurovascularly guided and/or neuro-metabolically guided training approach, the AI-powered computation unit 120 may process multiple streams of physiological data with minimal delay. Wireless protocols like ANT+ are optimized for low-latency transmission of biometric data, ensuring that information such as SpO₂, HRV, and CGM, blood pressure readings arrive quickly enough for the AI to make immediate protocol adjustments. This capability is vital for maintaining safety, for instance, triggering an emergency hyperoxic burst if a user's oxygen saturation or metabolic markers cross unsafe thresholds.

Wireless ingress 220 also enables interoperability and scalability. Because it uses industry-standard protocols, the device may pair with a wide range of commercially available wearables from different manufacturers. This not only future-proofs the system against evolving sensor technology but also allows for flexible integration into broader health ecosystems, including mobile apps, remote monitoring platforms, and cloud-based analytics. Furthermore, wireless connectivity facilitates secure data transfer to external devices for session logging, progress tracking, and clinician review, extending the system's utility beyond the training session itself.

Controller 150 serves as the central operational unit that manages the delivery of oxygen streams to the user in accordance with the training protocol. It acts as the intermediary between the AI-powered computation unit 120 and the oxygen generation hardware (e.g., the digitally controlled PSA system 130), executing precise adjustments to oxygen concentration, interval timing, and gas flow direction. Based on real-time inputs, such as SpO₂, HRV, CGM, and blood pressure readings, as well as the reading from the oxygen sensor 210, the controller 150 may rapidly switch between hypoxic and hyperoxic gas streams, adjust flow rates, or initiate emergency safety responses like a high-SpO₂ burst to stabilize the user. This real-time responsiveness ensures both optimal training effectiveness and user safety throughout each session.

In the disclosed IHHT device 100, the controller 150 may be implemented as an embedded system within the device chassis, integrating both hardware and software components. On the hardware side, the controller 150 may interface with actuators such as solenoid valves, flow regulators, and sensors embedded in the PSA system 130. On the software side, the controller 150 may run firmware capable of interpreting commands from the AI computation unit 120, translating high-level protocol adjustments into precise mechanical actions. This design allows the controller 150 to handle both routine session management, such as interval transitions, and urgent, high-priority commands triggered by abnormal physiological data.

It should be noted that, in the disclosed IHHT device, the controller 150 may be configured for closed-loop operation, meaning it continuously receives live sensor feedback and modifies the output in real time. This makes it a key enabler of the neurovascularly guided and/or training approach, where protocol parameters are dynamically tuned based on the user's combined autonomic vascular and/or metabolic state. In addition, the controller 150 may store protocol presets, execute fail-safe routines in the event of sensor dropout, and manage wireless communications with peripheral devices, further expanding its role as the operational heart of the IHHT device.

Optionally, remote and cloud monitoring 250 may be provided for the disclosed IHHT device 100 to enable real-time and historical session data to be securely transmitted from the device to external platforms, such as clinician dashboards, mobile applications, or cloud-based analytics services. This capability may allow healthcare providers, coaches, or researchers to observe a user's training performance and physiological responses without being physically present. Key metrics like SpO₂ trends, HRV patterns, CGM, blood pressure readings, and protocol adjustments may be streamed live or synced after a session, enabling timely feedback, safety oversight, and long-term progress tracking.

From a technical standpoint, remote/cloud monitoring relies on the device's wireless ingress 220 and outbound connectivity. Physiological data captured via Bluetooth or ANT+ from wearable sensors is processed by the computation unit 120, then securely encrypted and transmitted to a cloud server via Wi-Fi, LTE, or another internet connection. Cloud infrastructure stores this information in structured formats, making it accessible for data visualization, AI-driven trend analysis, and integration with electronic health records (EHRs). This architecture also supports asynchronous review, so multiple stakeholders, such as physicians, physiologists, or athletic trainers, can access the same dataset from different locations.

In some embodiments, beyond oversight, cloud monitoring 250 may enhance the adaptiveness and personalization of IHHT protocols. With aggregated historical data, the AI mode included in the computation unit 120 may identify long-term trends in a user's neurovascular and/or neuro-metabolic responses, enabling protocol refinements across weeks or months rather than just within a single session. In addition, cloud-based systems may issue proactive alerts, such as recommending a training pause if a user's physiological patterns suggest excessive strain, providing another layer of safety. This approach not only supports individualized care but also facilitates large-scale research studies by pooling anonymized data across many users to uncover broader insights into hypoxia-hyperoxia training outcomes.

The computation unit 120 in the IHHT device may function as the device's “AI brain”, integrating hardware, firmware, and software components to receive, process, and act upon real-time physiological data. Its primary role is to execute the adaptive control logic that enables neurovascularly and/or neuro-metabolically guided training, continuously analyzing incoming sensor data such as SpO₂, HRV, CGM, and blood pressure values and perfusion index to determine the optimal oxygen concentration, interval timing, and training intensity for the user.

Physically, the computation unit 120 may include one or more processors (CPUs or microcontrollers) connected to system memory, persistent storage, and I/O interfaces. These processors execute the AI algorithms, manage communication with peripheral devices, and control the timing and switching of the hypoxic and hyperoxic gas streams. Wireless interfaces such as Bluetooth, ANT+, or Wi-Fi may be integrated into the unit (or coupled to the unit) for sensor data ingestion and cloud connectivity. In some embodiments, the computation unit 120 may also feature a dedicated neural processing engine or GPU for accelerating machine learning model inference in real time.

In some embodiments, the computation unit 120 may run embedded firmware and higher-level software modules responsible for multi-modal sensor fusion, combining data from different physiological sources into a unified, real-time physiological stress score. This score reflects both the autonomic nervous system's state (via HRV and SpO₂) and the vascular and metabolic state (via CGM and/or blood pressure), enabling a closed-loop feedback system.

In some embodiments, the IHHT device 100 operates as a thin-client actuator. That is, the computation unit 120 on the device is not responsible for storing data, or any other type of logic. This data is stored in the cloud, as it is received through the mobile device such as a smartphone, and the protocols and instructions are sent from the mobile device or cloud. The computation unit 120 on the device is simply responsible for following the instructions sent from the mobile device or cloud. For example, a mobile application (e.g., on an iPhone) acquires physiological data and relays it to a cloud service that computes the session protocol in real time, target FiO₂ trajectories, interval timings, rescue triggers, and safety thresholds. The cloud returns time-stamped, authenticated control instructions to the mobile device, which forwards them over a secure short-range link to the device. The on-device computation unit simply parses these messages and drives valves, compressors, and flow controllers accordingly; it does not persist user data or host protocol-selection logic, retaining only transient buffers needed to execute current commands.

In some embodiments, machine learning models, such as neural networks, decision trees, or regression models, may be included in the computation unit 120 or in the mobile device or cloud. The model is mainly used to predict the user's optimal adaptive zone and dynamically adjust the training protocol. In some embodiments, the model may be pre-trained on general population data, then refined with an individual's historical data to create a personalized profile over time.

The exemplary machine model may be any one of a regression model (e.g., linear regression, logistic regression, or polynomial regression), decision tree, random forest, support vector machine, Naïve Bayes model, k-means cluster, or neural network (e.g., feed-forward networks, multilayer perceptron networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). In some embodiments, the machine learning model includes a dimensionality reduction component for visualizing data, the dimensionality reduction component comprising any of a principal component analysis (PCA) component, or a T-distributed Stochastic Neighbor Embedding (t-SNE). In some embodiments, the machine learning model includes a neural network or a random forest, which is not limited in the present disclosure.

In some embodiments, the machine learning model includes a large language model (LLM), a generative AI, a combination thereof, or any derivative model thereof. In some embodiments, the LLM or generative AI model or another different model may keep communications with a user under the training besides the adaptive adjustment of the training process described above. For example, based on the sensor data, the LLM or generative AI model may consistently communicate with a user under the training to guide the user to control and adjust the breath (e.g., take deeper breaths at some timepoints) and the tempo (take different stride lengths at different timepoints) during the training process.

In some embodiments, the machine learning model includes one or more parameters, such as hyperparameters and/or model parameters. Hyperparameters are generally established prior to training. Examples of hyperparameters include the learning rate, depth or leaves of a decision tree, number of hidden layers in a deep neural network, number of clusters in a k-means cluster, a penalty in a regression model, and a regularization parameter associated with a cost function. Model parameters are generally adjusted during training. Examples of model parameters include weights associated with nodes in layers of the neural network, variables and thresholds for splitting nodes in a random forest, support vectors in a support vector machine, and coefficients in a regression model. The model parameters of the machine learning model are trained (e.g., adjusted) using the training data to improve the power of the machine learning model.

In some embodiments, the training data used for the training of the machine learning model(s) may include historical training and/or testing data obtained from a lot of users with different health conditions. In some embodiments, more than one machine learning model may be included in the disclosed adaptive training system. For example, at least one machine learning model may be used to determine a training protocol, and at least another machine learning model may be used to dynamically adjust a training protocol being used by a user.

In some embodiments, the computation unit 120 or the AI model included herein may compute a unified neurovascular and/or neuro-metabolic stress score for a user during a training session, and thus acquires a plurality of physiological signals in real time as described above. These signals may be preprocessed by applying digital filtering to remove noise and motion artifacts, normalizing each signal relative to a baseline established from historical user data, and optionally applying temporal smoothing (e.g., moving average or exponential smoothing) to reduce short-term variability while preserving trend data. In some embodiments, the computation unit 120 may further include certain components or modules that extract a plurality of features from each preprocessed signal. For example, from SpO₂ data, the components or modules may extract absolute oxygen saturation, deviation from baseline, and rate of change; from HRV data, the components or modules may extract time-domain metrics (e.g., root mean square of successive differences (RMSSD), standard deviation of NN intervals (SDNN)), frequency-domain metrics (e.g., low frequency/high frequency (LF/HF) ratio), and short-term variability; and from CGM data, the components or modules may extract instantaneous glucose level, magnitude of excursion from baseline, and recovery slope following a hypoxic event and from blood pressure data, the components or modules may discriminate benign tachycardia from risky responses.

In some embodiments, the extracted features may be normalized to a common numerical range and assigned weighting factors based on predetermined rules, clinical relevance, or personalization parameters derived from prior user sessions. In some embodiments, oxygenation features may be weighted more heavily when hypoxic thresholds are approached, HRV features may be weighted more heavily for early detection of autonomic stress, and CGM and/or blood pressure features may be weighted more heavily for detection of metabolic strain and recovery rate.

In some embodiments, the normalized and weighted features may be fused into the unified neurovascular and/or neuro-metabolic stress score using a fusion algorithm, which may include: (i) a weighted sum calculation; (ii) a rule-based decision tree; (iii) a statistical model; or (iv) a machine learning model such as a random forest, gradient boosting machine, or neural network trained on labeled datasets where stress state is known.

In some embodiments, the unified neurovascular and/or neuro-metabolic stress score may be updated at a fixed sampling interval (e.g., between one second and five seconds) and is compared to adaptive thresholds to determine a training action or may be updated in real-time when there is any detected change (over a predefined threshold) of physiological data for dynamic adjustment of an ongoing training session. If the score is within a first threshold range, the current training protocol is maintained; if the score is within a second, higher threshold range, the IHHT device controller may reduce hypoxic intensity or increase recovery interval duration; and if the score exceeds a third threshold range, the IHHT device controller may initiate an emergency hyperoxic burst.

In some embodiments, when the computation unit 120 determines an adjustment is needed, the computation unit 120 may communicate with the controller 150 to execute mechanical actions, such as opening or closing valves, switching between hypoxic and hyperoxic streams, or initiating an emergency hyperoxic burst. The computation unit 120 may also push protocol updates to the device's local display or mobile application, ensuring that the user or remote coach is informed of changes in real time.

Additionally or alternatively, the computation unit 120 may further compute a metabolic flexibility biomarker (“MFB”) from a user's CGM time-series in response to a standardized hypoxic step. In some embodiments, the biomarker may be computed during a standardized hypoxic challenge (e.g., defined FiO₂ and duration) delivered when motion is low and sensor quality flags are adequate. In some embodiments, the computation unit 120 may map raw features to a unitless MFB score (e.g., 0-1, where higher=better flexibility) using a weighted combination.

In some embodiments, the computation unit 120 may consult the biomarker-linked guardrails while dosing for hypoxia. If excursion exceeds a user-specific limit or recovery speed falls below a threshold, the device may automatically (i) raise the minimum FiO₂, (ii) shorten the current or next hypoxic interval, (iii) extend recovery, and/or (iv) trigger a brief hyperoxic rescue burst. Progression to the next step is withheld until a recovery is met. Rate limiters and hysteresis may prevent oscillation.

In some embodiments, the computation unit 120 may maintain a trend of MFB and adjust starting parameters for subsequent sessions: improved MFB for recent sessions permits small, bounded dose escalations (e.g., +<10% hypoxic duration, −≤2 pp FiO₂), whereas deteriorating MFB tightens guardrails (shorter hypoxia, longer recovery, higher floor FiO₂). All adjustments may remain within clinician-approved bounds. In addition, certain hard safety limits may not be relaxed.

In some embodiments, if CGM is unavailable or low-confidence, the computation unit 120 may fall back to surrogate biomarkers (e.g., HRV recovery and/or FiO₂), apply conservative defaults, and flag the session. A clinician portal may review biomarker histories, set allowable ranges, and lock minimum thresholds. All computations, limits, and overrides may be versioned and auditable.

In some embodiments, the computation unit 120 may include or be coupled to storage 260 for both transient and long-term data. Short-term buffers may be used for high-speed processing during a session, while non-volatile memory may store historical training records, AI model parameters, and firmware. Security protocols, such as encrypted data transmission and secure boot, may be used to protect user information, particularly when transmitting data to remote/cloud monitoring systems.

In essence, the computation unit 120 transforms the IHHT device 100 from a static oxygen generator into an intelligent, adaptive training system, capable of understanding a user's unique neurovascular and/or neuro-metabolic signature, making split-second adjustments, and learning over time to deliver safer, more effective, and more personalized training outcomes.

While not shown, in some embodiments, the IHHT device 100 further includes certain safety monitors. In some embodiments, these safety monitors may supervise delivery independent of the control model. The monitors may enforce a minimum SpO₂ floor, cap hypoxic exposure within a rolling window, and detect leaks/occlusions by comparing commanded versus measured flow and/or pressure. On violation, the device may transition to a protective state (e.g., hyperoxic or ambient supply), alert the user, and record a timestamped fault event. Watchdog timers may verify sensor freshness and valve response, and monitored thresholds and actions may be clinician-configurable within prescribed limits.

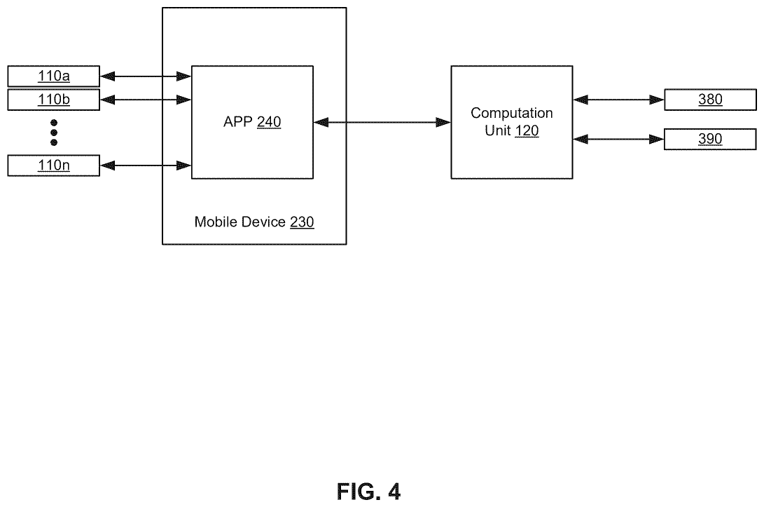

In the following, some specific hardware components, such as the digitally controlled PSA system 130 and buffering reservoir 140, are further described in detail. Specifically, FIG. 3 illustrates an example digitally controlled PSA system 300, according to some embodiments, and FIG. 4 illustrates an example buffering reservoir 400, according to some embodiments.

### Example of Digitally Controlled PSA System

As described earlier, in some embodiments, the disclosed IHHT device includes a digitally controlled PSA system 130 configured to selectively generate a hypoxic gas stream and a hyperoxic gas stream within a single integrated system. PSA systems may include molecular sieves as the medium where gas molecules adhere and detach based on controlled pressure variations, which is a gas separation process that utilizes the differing affinities of gas molecules for an adsorbent material under varying pressure. Essentially, it works by selectively adsorbing (attaching to a surface) certain gases at higher pressures and then releasing them at lower pressures. This cyclic process allows for the separation and purification of gases like hydrogen, nitrogen, and oxygen, which can then be used to generate a hyperoxic gas stream and a hypoxic gas stream.

As illustrated in FIG. 3, the digitally controlled PSA system 130 may include a couple of adsorption towers 350a and 350b to produce both a nitrogen-enriched, oxygen-depleted gas stream and an oxygen-enriched gas stream having a fractional inspired oxygen concentration (FiO₂) exceeding 80%. Briefly, when the adsorption tower 350a is in the adsorption stage, zeolite may preferentially capture nitrogen, so the product leaving the adsorption tower A is an oxygen-rich hyperoxic gas stream. At the same time, while the adsorption tower 350a is in the adsorption stage, the adsorption tower 350b is in the desorption stage or regeneration stage, where it is depressurized to desorb the trapped nitrogen, so as to generate a nitrogen-rich hypoxic gas stream. The two adsorption towers then swap roles every few seconds, so that the hypoxic and hyperoxic gas streams may be continuously provided by the alternated outputs of the two gas streams.

It should be noted that, while in the above example, the molecular sieve media is selected to preferentially adsorb nitrogen molecules, the present disclosure is not limited thereto. In some embodiments, a molecular sieve media is selected to preferentially adsorb oxygen molecules, thereby allowing nitrogen-rich gas stream to be desorbed while the oxygen-rich gas stream is adsorbed.

In some embodiments, ambient air enters the IHHT device 100 through two separate inlet points, each feeding a dedicated filter 310a or 310b. These filters 310a/310b remove particulate matter and contaminants, ensuring that only clean air reaches the compressor 320. Once filtered, the air is drawn into the compressor 320, where its pressure is increased to the operational level required for PSA separation. The high-pressure air then passes through a heat exchanger 330, which may condition the air to a proper temperature, preventing excess heat or cold from affecting the adsorption process. From the heat exchanger 330, the conditioned compressed air flows into a central control valve assembly 340.

The central control valve assembly 340 acts as the traffic director of the system, determining whether the incoming compressed air is routed into absorption tower one or absorption tower two. As described earlier, each tower 350a or 350b contains zeolite molecular sieve material, such as the one that selectively adsorbs nitrogen from the incoming air, leaving an oxygen-rich gas mixture, which is then purged from the tower and vented through dedicated equilibrium canister 360a or 360b. An equilibrium canister is a small, rigid, oxygen-clean plenum installed on the hyperoxic PSA product line. It equalizes (1) pressure ripple and (2) O₂ concentration ripple coming out of the PSA towers, so the downstream selector valve always sees a stable, high-purity (e.g., >80% FiO₂) reservoir. It also provides short “surge” capacity for brief rescue bursts without purity sag. The outlet of the equilibrium canister is coupled to a selector valve 365 that alternately directs the hyperoxic gas stream to a buffering reservoir 140a or optionally to the central control valve assembly 340, which can be then re-routed to a adsorption tower 350a or 350b that is generating the oxygen-rich gas stream, so that the hyperoxic gas stream can be reused to improve the efficiency of the whole PSA system.

While one tower is actively producing hyperoxic air, the other is in a regeneration cycle. During regeneration, nitrogen-rich exhaust is purged from the tower and vented through dedicated nitrogen mufflers 370a or 370b, which are engineered to reduce cyclic PSA/valve noise and breath-path hiss without adding noticeable resistance or shedding fibers into the patient circuit. The muffler may include a reactive stage having one or more Helmholtz resonators tuned to attenuate cyclic pressure and concentration ripple from a pressure-swing adsorption cycle, and an absorptive stage including a micro-perforated panel disposed within an expansion chamber to attenuate broadband valve noise. The outlets of the mufflers is coupled to a selector valve 375 that alternately directs the hypoxic gas stream to a buffering reservoir 140b or optionally to the central control valve assembly 340, which can be then re-routed to a adsorption tower 350a or 350b that is generating the nitrogen-rich gas stream, so that the hypoxic gas stream can be reused to further improve the efficiency of the whole PSA system. In some embodiments, the routing of the selector valve 375, as well as the selector valve 365 mentioned earlier, can be automatically controlled by the computation unit 120 and/or further manually adjusted under certain circumstances, which are not limited in the present disclosure.

After the routing through the selector valve 365 or 375, each hyperoxic or hypoxic gas stream may be guided into a respective buffering reservoir 140a or 140b, both of which are internal reservoirs within the chassis 105. The buffering reservoir 140a/140b may include an internal, chassis-integrated reservoir that acts as a pneumatic low-pass filter between the PSA system 130 and the breathing interface 160. It smooths flow and pressure transients during phase changes and inspiratory peaks, shortens the effective pneumatic path versus external bags and long hoses, and thereby improves control responsiveness and user comfort. In some embodiments, the reservoir may include embedded pressure (and optional flow/temperature) sensing for closed-loop supervision, a pressure-relief pathway to prevent over-pressurization, and, optionally, certain service features such as an antimicrobial liner and accessible hatch. The integrated placement reduces external leak points, footprint, and setup steps while providing stable delivery of the commanded FiO₂. It should be noted that, while two buffering reservoirs are included in the disclosed digitally controlled PSA system 130, in some embodiments, only one buffering reservoir is included. For example, in some embodiments, there is only one buffering reservoir for the hypoxic gas stream, but there is no buffering reservoir for the hyperoxic gas stream.

The switching valve 380 is a computer-controlled manifold that (i) selects between the hypoxic and hyperoxic gas streams from the buffering reservoir 140a or 140b, and (ii) in some operation modes proportionally blends a selected stream with ambient air to achieve a commanded FiO₂ before delivery to a single output 390 before delivery to the breathing interface 160. It is specified for fast actuation (e.g., sub-100 ms), low dead volume, and low leakage, and may include position feedback and downstream flow/pressure sensing (e.g., flow sensor 210) for closed-loop setpoint control. One-way isolation may be used to prevent cross-flow between paths. In some embodiments, certain firmware may be used to apply hysteresis and rate limits to avoid oscillation during frequent transitions. In addition, a fail-safe bias to a breathable state (e.g., ambient or hyperoxic) may be further imposed on detected fault or power loss. Calibration routines verify valve linearity and blend accuracy over time.

In some embodiments, the switching valve 380 may be further used as a mixing valve, allowing a mixing of both hypoxic and hyperoxic gas streams supplied by the buffering reservoirs 140a and 140b. For example, for timely adjustment, a brief mixture of hypoxic gas stream into a main hyperoxic gas stream may allow the delivered gas mixture to adjust a little without necessarily switching between two dedicated gas streams.

While not shown in FIG. 3, in some embodiments, to manage continuous gas production during periods of low or paused user demand, the digitally controlled PSA system 130 further includes one or more pressure relief mechanisms. In one example, an internal buffering reservoir 140 may be fluidly coupled to a first pressure relief valve configured to vent excess gas to the ambient atmosphere when a pressure within the reservoir exceeds a predetermined threshold. In some embodiments, a gas output pathway is fluidly coupled to a second pressure relief valve configured to vent excess gas to the ambient atmosphere when a pressure within the pathway exceeds a predetermined threshold. The provision of these pressure relief valves maintains safe and stable operation of the digitally controlled PSA system 130 and prevents over-pressurization of system components.

In the disclosed digitally controlled PSA system 130, the architecture overcomes the “two-mask restriction” inherent in other existing PSA-based IHHT systems and, for the first time, enables full digital control of hypoxic and hyperoxic delivery through a single user interface. In an example embodiment illustrated in FIG. 4, a computation unit 120 is configured to receive commands and protocol instructions from a user's mobile device 230 via a wireless communication link (e.g., Bluetooth). The mobile device application 240 is configured to execute personalized training protocols that adapt in real time based on physiological data received from one or more wearable sensors 110a, 110b, . . . , 110n, including, for example, sensors for blood oxygen saturation (SpO₂), heart-rate variability (HRV), continuous glucose monitoring, blood pressure, and perfusion index (which may be directly sent to the computation unit 120 or through the mobile device 230). The application 240 is further configured to transmit control commands to the computation unit 120 (and/or controller 150 included therein) to adjust hypoxic intensity, flow rate, or to operate a switching valve 380 to deliver brief, intra-session hyperoxic bursts for safety purposes and/or protocol optimization.

In some embodiments, adjustment of hypoxic intensity is achieved by the computation unit 120 transmitting digital control signals to the digitally controlled PSA system 130 to modify one or more operational parameters, such as cycle time and airflow rate. To ensure precision, an oxygen sensor 210 is disposed within a fluid pathway leading to a single user outlet (e.g., after the switching valve 380). The computation unit 120 is configured to receive real-time oxygen concentration data from the oxygen sensor 210, compare the received concentration to a target concentration specified by the active protocol, and execute fine-tuning adjustments to the operational parameters of the digitally controlled PSA system 130 in a closed-loop manner to maintain the target concentration.

### Example of Buffer Reservoir

In the following description, an example buffering reservoir 140 is further described by taking an expandable buffer bag having an accordion-style geometry as an example. However, it is to be noted that buffering reservoir 140 may take other forms. For example, in some embodiments, the buffering reservoir 140 may be a rigid cylinder under certain configurations.

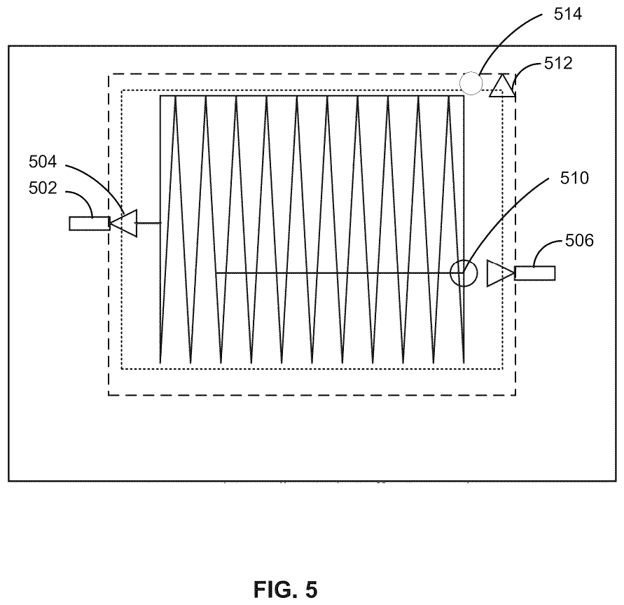

In the illustrated embodiment in FIG. 5, the expandable body may be formed from a flexible, gas-impermeable material configured to withstand repeated expansion and contraction cycles without material fatigue, deformation, or leakage. The accordion-style geometry is configured to permit controlled volumetric expansion to accommodate incoming flow surges and controlled contraction to maintain downstream output flow during periods of reduced upstream supply.

In some embodiments, the buffering reservoir 140 includes an inlet port 502 in fluid communication with an output of the equilibrium canister 360 or directly with an adsorption tower 350. In some embodiments, the integrated buffering reservoir 140 may further include an inlet check valve 504 positioned between the output of the equilibrium canister 360 or the adsorption tower 350. The inlet check valve 504 is configured to permit gas flow into the buffering reservoir while preventing reverse flow toward the upstream generation components. This unidirectional control maintains separation between hypoxic and hyperoxic gas streams and prevents pressure backflow from the buffering reservoir into the equilibrium canister 360 or the adsorption tower 350 during pressure switching or compressor idle cycles.

An outlet port 506 of the buffering reservoir is fluidly coupled to the switching valve 380 that is digitally controlled. The switching valve 380 may allow gas flow to exit the buffering reservoir toward the delivery interface while preventing ingress of ambient air or exhaled breath back into the reservoir. This maintains gas purity and protects internal components from contamination or moisture intrusion during user inhalation and exhalation cycles. The buffering reservoir 140 is thereby operable to smooth flow transitions resulting from switching events, compressor cycling, or blending ratio adjustments.

In certain embodiments, a flow sensor 510 may be disposed at or near the outlet port 506 of the buffering reservoir and is configured to measure the volumetric flow rate of the gas delivered to the switching valve 380 and further to the breathing interface. Data from the flow sensor 510 may be communicated to the computation unit 120 or controller 150 to monitor user breathing patterns, verify protocol adherence, and adjust oxygen delivery parameters in real time.

In some embodiments, the buffering reservoir 140 may further incorporate a relief valve 512 configured to vent excess gas pressure if the internal reservoir pressure exceeds a predetermined threshold. The relief valve 512 may be spring-loaded or otherwise biased to open automatically under overpressure conditions, thereby preventing damage to the buffering reservoir, downstream components, or the breathing interface. In some embodiments, the relief valve 512 is positioned and oriented to vent gas safely away from the user and sensitive electronics. In some embodiments, the buffering reservoir 140 may additionally include a pressor sensor 514 to monitor the pressure before the release.

In the disclosed IHHT device 100, the integration of the buffering reservoir 140 within the device chassis is configured to reduce device footprint, improve portability, and enhance durability by physically shielding the buffering reservoir from accidental puncture, kinking, or environmental damage. The internal placement also eliminates the need for external, inflatable bag-type reservoirs commonly used in other existing IHHT devices, thereby simplifying user setup and reducing potential points of failure.

In some embodiments, the buffering reservoir 140 is operatively controlled in coordination with the computation unit 120 or controller 150, such that gas flow characteristics within the reservoir may be dynamically adjusted to meet real-time protocol requirements determined from physiological data inputs, including but not limited to SpO₂, HRV, CGM and blood pressure data.

### Example Implementation

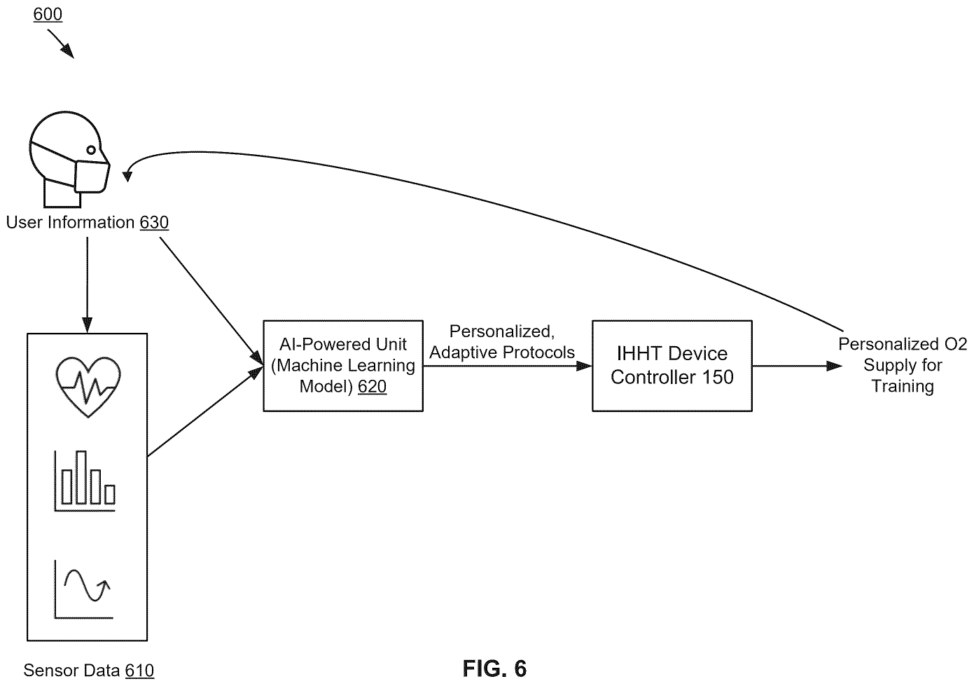

Referring now to FIG. 6, an example method 600 for operating the AI-powered IHHT device 100 is described, in accordance with an embodiment. Briefly, user information 630 may be first collected. For example, a person may log into the computation system of the device (e.g., through the mobile application 240 of the mobile device 230). Alternatively, if the device is dedicated to personal use, the person may be recognized automatically by default. Once the user information 630 is collected, it is provided, together with sensor data 610 obtained from one or more physiological sensors, to an AI-powered unit (machine learning model) 620.

The AI-powered unit 620 is configured to identify a personalized, adaptive training protocol for the user. The identified protocol may be the same as a previously used protocol for that user or a new protocol derived from population-level data for users with similar personal information, such as age, weight, or health status. In some embodiments, the computation unit 120 of the device 100 may also allow the user to select from a set of predefined protocols. Once the protocol is determined, it is transmitted from the AI-powered unit 620 to the IHHT device controller 150, which manages the oxygen generation system to deliver a personalized O₂ supply for training according to the specified adaptive protocol.

During the training session, the user may be monitored continuously through one or more wearable or non-wearable sensors, generating sensor data 610 in real time. This sensor data may include parameters such as SpO₂, HRV, CGM, blood pressure, and other possible physiological metrics relevant to training adaptation. The real-time data is sent back to the AI-powered unit 620, which evaluates whether adjustments to the current protocol are necessary. In one example, a unified neurovascular and/or neuro-metabolic stress score from SpO₂, HRV, CGM, and blood pressure data may be computed in the computation unit, which may be used to determine if an adjustment is necessary. If an adjustment is warranted, the AI-powered unit 620 generates an updated protocol and sends it to the IHHT device controller 150, which then alters the oxygen supply parameters accordingly. This closed-loop process may be repeated throughout the session, enabling continuous optimization of the training protocol in real time. In another embodiment, a metabolic flexibility biomarker may be similarly monitored through the sensed data for protocol adjustment.

In some embodiments, the AI-powered unit 620 may also generate personalized recommendations based on the analysis of past training sessions combined with user information 630. For example, older individuals may be advised to follow less intense training regimens, while younger individuals may be guided toward more demanding protocols. Additionally, based on historical and session-specific data, the AI-powered unit 620 may recommend complementary activities, such as targeted exercise routines or lifestyle adjustments, to further enhance the effectiveness of IHHT-based training.

### Personalization

In some embodiments, the computation unit 120 may maintain a per-user profile that is updated across sessions from time-aligned physiological inputs (e.g., SpO₂ trajectories, HRV suppression/recovery, and kinetics), device-side circuit measurements (e.g., flow/pressure dynamics, leak), and protocol metadata (e.g., hypoxic/hyperoxic durations, FiO₂ setpoints, rescue events). The computation unit may derive summary features per interval and per session (e.g., time-to-SpO₂-floor, maximum desaturation rate, HRV nadir and recovery half-time, glucose excursion area-under-curve and recovery slope) and update a vector of per-user response parameters using a bounded adaptive estimator (e.g., exponentially weighted moving average with outlier rejection and confidence weighting by data quality flags).

In some embodiments, the computation unit 120 may map learned parameters to individualized safety thresholds and control bounds, including but not limited to minimum SpO₂ floor, maximum permitted desaturation rate, maximum hypoxic exposure per rolling window, maximum allowed CGM excursion, and minimum required recovery slope before advancing dose. Personalization may tighten guardrails for sensitive users (e.g., lower SpO₂ floor, shorter hypoxic windows when glycemic sensitivity is high) and relax within clinician-approved limits when repeated sessions demonstrate improved tolerance.

For each new session, the computation unit 120 may select initial FiO₂ targets, interval durations, ramp rates, and rest periods by referencing the per-user parameters and recent biomarker trends. For example, when the hypoxic tolerance index and glycemic sensitivity both improve for N consecutive sessions, the next session may increase hypoxic duration by a bounded step (e.g., <10%) and/or lower target FiO₂ by a small decrement (e.g., <1-2 percentage points), subject to guardrails. Conversely, if CGM excursion exceeds a user-specific limit or HRV recovery half-time worsens beyond a threshold, the schedule may be automatically de-escalated (shorter hypoxic intervals, longer recovery, higher minimum FiO₂) and a hyperoxic rescue burst trigger is made more sensitive. During a session, the controller may adjust the schedule in real time from a neurovascular and/or neuro-metabolic stress score or biomarker score while the computation unit logs cause-and-effect tuples (state, action, response), as described above. Post-session, these tuples may update the per-user parameters and optionally a lightweight personalization layer so that future sessions start closer to the user's current capacity.

System and/or Computer Embodiments

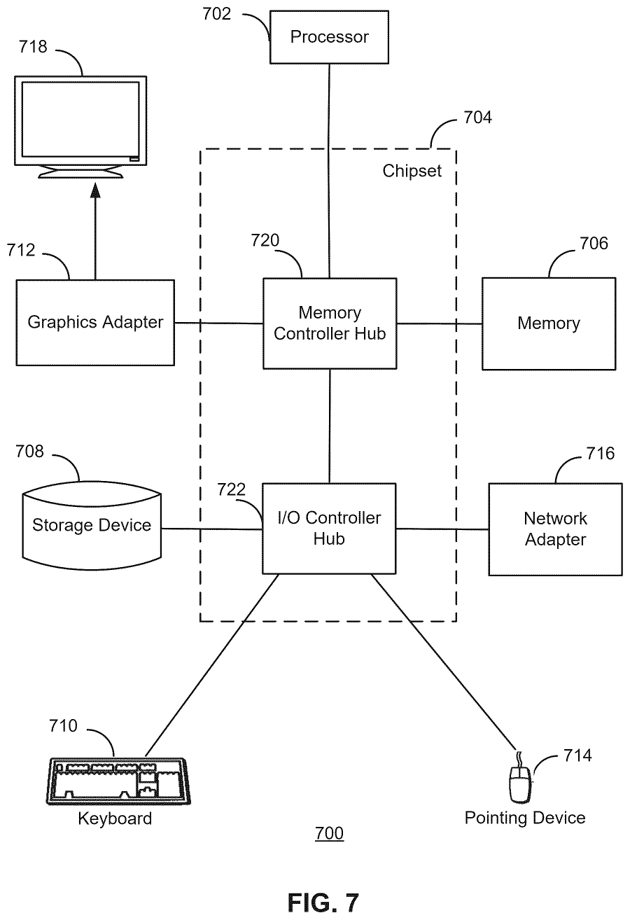

FIG. 7 depicts an example computing device 700 for implementing systems and methods described in reference to FIGS. 1-6. Examples of a computing device may include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, edge devices, IoT devices, and the like. In some embodiments, the computing device 700 may operate as an AI-powered unit. Thus, the computing device 700 may train and/or deploy machine learning models for monitoring IHHT training sessions.

In some embodiments, the computing device 700 includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input interface 714, and a network adapter 716 are coupled to the I/O controller hub 722. Other embodiments of the computing device 700 have different architectures.

The storage device 708 is a non-transitory computer-readable storage medium such as a hard drive, compact disk read-only memory (CD-ROM), DVD, or a solid-state memory device. The Memory 706 holds instructions and data used by the processor 702. The input interface 714 is a touch-screen interface, a mouse, a trackball, or other types of input interface, a keyboard, or some combination thereof, and is used to input data into the computing device 700. In some embodiments, the computing device 700 may be configured to receive input (e.g., commands) from the input interface 714 via gestures from the user. The graphics adapter 712 displays images and other information on the display 718. The network adapter 716 couples the computing device 700 to one or more computer networks.

The computing device 700 is adapted to execute computer program modules for providing the functionality described herein. As used herein, the term “module” refers to computer program logic used to provide the specified functionality. Thus, a module may be implemented in hardware, firmware, and/or software. In one embodiment, program modules are stored on the storage device 708, loaded into the memory 706, and executed by the processor 702.

The types of computing devices 700 may vary from the embodiments described herein. For example, the computing device 700 may lack some of the components described above, such as graphics adapters 712, input interface 714, and displays 718. In some embodiments, a computing device 700 may include a processor 702 for executing instructions stored in a memory 706.

The methods disclosed herein may be implemented in hardware or software, or a combination of both. In one embodiment, a non-transitory machine-readable storage medium, such as the one described above, is provided, the medium comprising a data storage material encoded with machine-readable data which, when using a machine programmed with instructions for using said data, is capable of displaying any of the datasets and execution and results of this disclosure. Such data may be used for a variety of purposes, such as patient monitoring, treatment considerations, and the like. Embodiments of the methods described above may be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices in a known fashion. The computer may be, for example, a personal computer, microcomputer, or workstation of conventional design.

Each program may be implemented in a high-level procedural or object-oriented programming language to communicate with a computer system. However, the programs may be implemented in assembly or machine language, if desired. In any case, the language may be a compiled or interpreted language. Each such computer program is preferably stored on a storage medium or device (e.g., ROM or magnetic diskette) readable by a general or special-purpose programmable computer, for configuring and operating the computer when the storage medium or device is read by the computer to perform the procedures described herein. The system may also be considered to be implemented as a computer-readable storage medium, configured with a computer program, where the storage medium so configured causes a computer to operate in a specific and predefined manner to perform the functions described herein.

The signature patterns and databases thereof may be provided in a variety of media to facilitate their use. “Media” refers to a medium that contains the signature pattern information of the present disclosure. The databases of the present disclosure may be recorded on computer-readable media, e.g., any medium that may be read and accessed directly by a computer. Such media include, but are not limited to: magnetic storage media, such as floppy discs, hard disc storage media, and magnetic tape; optical storage media such as CD-ROM; electrical storage media such as RAM and ROM; and hybrids of these categories, such as magnetic/optical storage media. One skilled in the art may readily appreciate how any of the presently known computer-readable mediums may be used to create a manufacture comprising a recording of the present database information. “Recorded” refers to a process for storing information on a computer-readable medium, using any such methods as known in the art. Any convenient data storage structure may be chosen, based on the means used to access the stored information. A variety of data processor programs and formats may be used for storage, e.g., word processing text files, database format, etc.

What is claimed is:

1. An intermittent hypoxia-hyperoxia training (IHHT) apparatus, comprising:
a main chassis;
a pressure swing adsorption (PSA) system housed within the chassis, the PSA system configured to generate a hyperoxic gas output and a hypoxic gas output;
a buffering reservoir housed within the main chassis, configured to receive the hypoxic gas output from the PSA system;
a single user output port;
a valve mechanism having a first inlet connected to an output of the buffering reservoir, a second inlet connected to the hyperoxic gas output of the PSA system, and an outlet connected to the single user output port; and
a computation unit configured to control the valve mechanism, thereby selecting whether hypoxic gas from the buffering reservoir or hyperoxic gas from the PSA system is delivered to the single user output port.

2. The apparatus of claim 1, further comprising a first pressure relief valve fluidly connected to the buffering reservoir, configured to vent hypoxic gas when a pressure within the buffering reservoir exceeds a predetermined threshold.

3. The apparatus of claim 2, further comprising a second pressure relief valve fluidly connected to the hyperoxic gas output, configured to vent hyperoxic gas to prevent over-pressurization of the PSA system.

4. The apparatus of claim 1, wherein the computation unit further comprises a wireless transceiver configured to receive commands from an external user device.

5. The apparatus of claim 4, wherein the commands instruct the computation unit to control at least one of the valve mechanism, a flow rate of the apparatus, or an oxygen concentration of the hypoxic gas output by digitally adjusting operational parameters of the PSA system.

6. The apparatus of claim 5, further comprising an oxygen sensor disposed in a fluid pathway leading to the single user output port, wherein the computation unit is further configured to receive a signal from the oxygen sensor and adjust the operational parameters of the PSA system based on the signal to achieve a target oxygen concentration.

7. The apparatus of claim 4, wherein the external user device is a mobile device executing a software application, and wherein the wireless transceiver operates on a Bluetooth protocol.

8. The apparatus of claim 1, wherein the buffering reservoir is selected from one or more of an expandable buffer bag or a rigid cylinder.

9. The apparatus of claim 1, wherein the PSA system is configured to generate a hyperoxic gas with a fractional inspired oxygen (FiO₂) concentration greater than 80%.

10. A method for providing adaptive IHHT training using the apparatus of claim 4, the method comprising:
receiving, at the external user device, a plurality of real-time physiological data streams from one or more wearable sensors monitoring a user;
processing, by the external user device, said physiological data streams to determine a need for a protocol adjustment;
transmitting, from the external user device to the computation unit of the apparatus via the wireless transceiver, a command to implement the protocol adjustment; and
executing, by the computation unit, the command to adjust the training being administered to the user.

11. The method of claim 10, wherein the plurality of real-time physiological data streams comprises one or more of blood oxygen saturation (SpO₂) data, heart-rate variability (HRV) data, blood pressure data, perfusion index, and continuous glucose monitoring (CGM) data.

12. The method of claim 10, wherein the protocol adjustment comprises a command to operate the valve mechanism to initiate an intra-session hyperoxic burst.

13. The method of claim 10, further comprising storing the physiological data streams and corresponding protocol adjustments in a cloud-based data store to longitudinally improve future training protocols for the user.

14. A system for intermittent hypoxia-hyperoxia training, comprising:
one or more sensors configured to obtain physiological data of a user under the IHHT training;
a computation unit configured to compute a score based on the physiological data of the user;
a gas generator configured to produce a nitrogen-enriched, oxygen-reduced hypoxic gas stream and an oxygen-enriched hyperoxic gas stream, wherein the gas generator is further fluidly connected to a buffering reservoir for buffering one or more of the hyperoxic gas stream and the hypoxic gas stream; and
a single breathing interface coupled to the buffering reservoir, wherein the computation unit further includes a controller operatively coupled to the gas generator to adjust a flow of at least one of the hyperoxic gas stream or the hypoxic gas stream during a training session responsive to the generated score.

15. The system of claim 14, wherein the physiological data includes one or more of SpO₂ data, HRV data, blood pressure data, perfusion index, and CGM data.

16. The system of claim 14, wherein the gas generator is a zeolite-based digitally controlled pressure swing adsorption (PSA) system.

17. The system of claim 14, wherein the buffering reservoir has an accordion geometry configured to smooth flow and pressure transients.

18. The system of claim 14, further comprising a wireless ingress configured to receive sensor data via Bluetooth low energy or ANT+ using authenticated and encrypted transport.

19. The system of claim 14, further comprising an oxygen sensor proximate to the breathing interface configured to ensure that an oxygen concentration delivered is at a target level at any point of the training session.

20. The system of claim 14, wherein the controller is further configured to initiate a brief hyperoxic rescue burst upon the score exceeding a predefined threshold.
